(19) United States
(12) Patent Application Publication
(10) Pub. No.: US 2012/0253188 A1
(43) Pub. Date: Oct. 4, 2012

(54) REDUCING RISK OF COMPLICATIONS ASSOCIATED WITH TISSUE ABLATION

(75) Inventor: George A. HOLLAND, Marblehead, MA (US)

(73) Assignee: UNIVERSITY OF ROCHESTER, Rochester, NY (US)

(21) Appl. No.: 13/434,750

(22) Filed: Mar. 29, 2012

Related U.S. Application Data
(60) Provisional application No. 61/469,010, filed on Mar. 29, 2011.

(51) Int. Cl. A61M 37/00; A61B 6/00; A61B 18/14

(52) U.S. Cl. 600/431; 606/41; 604/20

(57) ABSTRACT

Methods and systems are described that reduce risks of hematologic, metabolic, and renal complications in a mammal, such as a human, undergoing tissue ablation. One such method includes inserting a probe into a mammal and ablating abnormal tissue in the mammal by emitting a first amount of energy from the probe. In some embodiments, after emitting the first amount of energy, a method can include denaturing proteins released from cells in the abnormal tissue by emitting a second amount of energy from at least one of the first probe or a second probe inserted into the mammal. Furthermore, some embodiments can be implemented such that during or after the emitting the energy from a probe, a composition is administered to the mammal in an effective amount to denature proteins released from cells in the abnormal tissue.

### CROSS-REFERENCE TO RELATED APPLICATIONS

This application claims the benefit of U.S. Provisional Application No. 61/469,010, filed Mar. 29, 2011, the entirety of which is incorporated herein by reference.

### FIELD

The present subject matter relates to methods for reducing risks of complications in a mammal undergoing tissue ablation.

### BACKGROUND

In many medical procedures, such as the treatment of benign or malignant tumors, it is important to remove the undesirable tissue without affecting the surrounding tissue. Tissue ablation is a technique used for the removal of undesirable tissue. Two types of tissue ablation, for example, are irreversible electroporation and cryoablation.

### SUMMARY

Despite the benefits of tissue ablation, various types of tissue ablation can lead to certain complications. Accordingly, in accordance with at least one of the aspects of the present technology, methods and features of the embodiments disclosed herein can be implemented to overcome various complications associated with tissue ablation.

As described herein, various embodiments relate to methods for reducing the risk of at least hematologic, metabolic, and renal complications following tissue ablation.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

Following tissue ablation in a mammal, such as by irreversible electroporation or cryoablation, damage to ablated cells results in the release of intracellular contents of the ablated cells (e.g., proteins, such as enzymes) that can lead to hematologic, metabolic, and renal complications for the mammal. Such complications can include disseminated intravascular coagulation (DIC), bleeding, and tumor lysis syndrome. A need exists for methods of reducing risks of such complications during and following tissue ablation.

Various embodiments of the present technology relate to methods for reducing a risk of, e.g., hematologic and metabolic complications in a mammal undergoing tissue ablation.

One aspect of the technology provides a method for reducing a risk of hematologic and metabolic complications in a mammal undergoing tissue ablation. The method can comprise inserting a first probe into a mammal and ablating abnormal tissue in the mammal by emitting a first amount of energy from the first probe.

Further, in accordance with some embodiments, the method can be implemented such that after emitting the first amount of energy, proteins released from cells in the abnormal tissue are denatured by emitting a second amount of energy from at least one of the first probe or a second probe inserted into the mammal. Furthermore, some embodiments can be implemented such that during or after the emitting the energy from a probe, a composition is administered to the mammal in an effective amount to denature proteins released from cells in the abnormal tissue. Additionally, some embodiments of methods disclosed herein can denature abnormal tissue by both emitting a second amount of energy and administering a composition to the mammal.

In some aspects of the technology, the ablating of abnormal tissue can be performed using electroporation that is at least partially reversible, or using irreversible electroporation (IRE). In some aspects, the ablating of abnormal tissue can be performed using cryoablation. In some aspects, ablation can occur through tissue heating by application of, e.g., radiofrequency or microwave energy.

In some aspects of the technology, the proteins released from cells in the abnormal tissue can be denatured by emitting a second amount of microwave energy or radiofrequency energy from at least one of the first probe or a second probe inserted into the mammal or by administering a composition.

In some aspects of the technology, the method can also involve monitoring heating of a region of tissue comprising the abnormal tissue during and/or after emitting the first amount of energy. Further, in some aspects, the method also involves monitoring heating of a region of tissue comprising the abnormal tissue during and/or after emitting the second amount of energy. In accordance with some embodiments, the monitoring can be performed with a temperature sensor placed in the mammal. The monitoring also can be performed by imaging such as computed topography or magnetic resonance imaging.

In accordance with some aspects of the technology, the proteins released from cells in the abnormal tissue can be denatured when the second amount of energy heats the proteins or target tissue to a temperature of between about 42° C. and about 60° C. In some embodiments, the second amount of energy can be applied for at least one hour or more. Further, in some aspects, the proteins released from cells in the abnormal tissue can be denatured nearly instantaneously when the second amount of energy heats the proteins or target tissue to a temperature of between at least about 62° C. and about 65° C. or greater.

In some aspects, the method can involve monitoring the denaturation of proteins released from a region of tissue comprising the abnormal tissue during and/or after emitting the first amount of energy.

In some aspects of the technology, the composition used to denature proteins released from cells in the abnormal tissue can be saline that is hypertonic relative to a concentration of saline in the mammal's circulation. In some aspects, the composition used to denature proteins released from cells in the abnormal tissue can be a denaturing agent comprising at least one of a chaotropic agent, a lyotropic agent, an organic denaturant, or a detergent.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 is a flowchart of a method for reducing a risk of hematologic and metabolic complications in a mammal undergoing tissue ablation according to an embodiment.

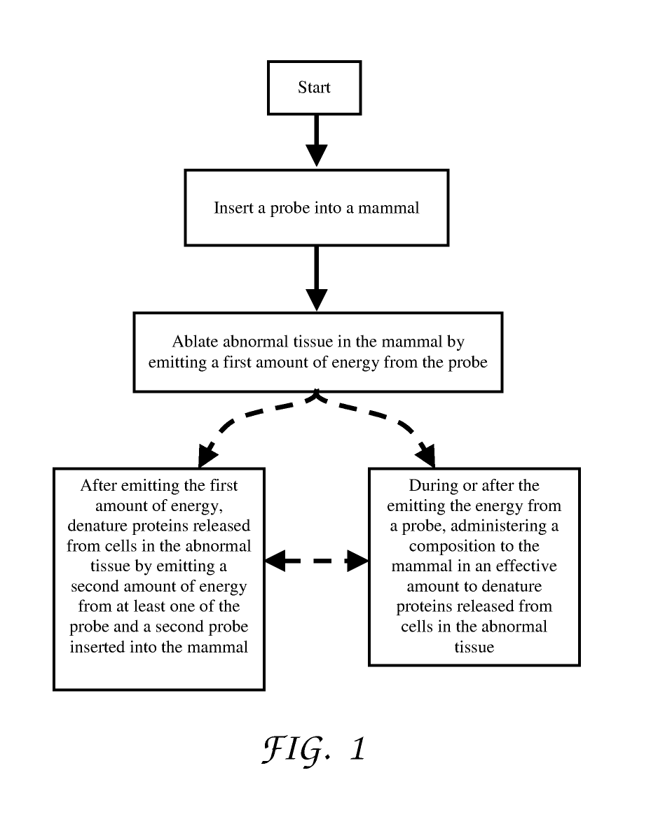

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

The foregoing description is provided to enable a person skilled in the art to practice the various configurations described herein. While the subject technology has been particularly described with reference to the various details, it should be understood that these are for illustration purposes only and should not be taken as limiting the scope of the subject technology. All publications, patents, and patent applications cited herein are hereby incorporated by reference for all purposes.

There may be many other ways to implement the subject technology. Various functions and elements described herein may be partitioned differently from those shown without departing from the scope of the subject technology. Various modifications to these methodologies will be readily apparent to those skilled in the art, and generic principles defined herein may be applied to other configurations. Thus, many changes and modifications may be made to the subject technology, by one having ordinary skill in the art, without departing from the scope of the subject technology.

As used herein, the singular forms “a,” “an” and “the” include plural references unless the content clearly dictates otherwise.

The term “about,” as used here, refers to +/−10% of a value.

Furthermore, to the extent that the terms “include,” “have,” or the like are used in the description or the claims, such terms are intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such as “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

By “mammal” is meant member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

Tissue Ablation

Tissue ablation refers to the removal of abnormal and/or undesirable tissue. The ablation of abnormal and/or undesirable tissue is performed in a controlled and focused way without affecting the surrounding, desirable, and/or healthy tissue. The term “tissue ablation” encompasses techniques such as, but not limited to, irreversible electroporation (IRE), radiofrequency ablation, interstitial laser ablation, focused ultrasound ablation, microwave ablation, and cryoablation. Tissue ablation techniques may be percutaneous or open procedures, and may be visualized or guided by imaging modalities such as ultrasound, fluoroscopy, x-ray tomography, stereotactic mammography, computed tomography (CT), or magnetic resonance imaging (MRI). Real-time imaging of the zone of ablated tissue is typically achieved with ultrasound, CT, or MRI.

Examples of disorders or conditions that can be treated using tissue ablation include, but are not limited to, benign and malignant tumors; cardiac arrhythmia such as atrial flutter, atrial fibrillation, supraventricular tachycardia, and ventricular arrhythmias; varicose veins; chronic pain for example in the lower (lumbar) back due to a herniated intervertebral disc, and benign prostatic hyperplasia (BHP). Examples of tumors that can be treated using tissue ablation are primary tumors in organs such as, but not limited to, breast, lung, liver, brain, pancreas, stomach, colon, kidney, and bone, as well as tumors that have metastasized to such organs. Tissue ablation used to treat tumors can be combined with chemotherapy, radiation therapy, and prior surgical resection.

Electroporation is defined as the phenomenon that makes cell membranes permeable by exposing them to certain electric pulses. See Weaver, J. C. and Y. A. Chizmadzhev, Theory of electroporation: a review. Bioelectrochem. Bioenerg., 1996, 41: p. 135-60.

The term “reversible electroporation” encompasses permeabilization of the cell membrane through the application of electrical pulses across the cell. In “reversible electroporation” the permeabilization of the cell membrane generally ceases after the application of the pulse, and the cell membrane permeability reverts to normal. The cell usually survives “reversible electroporation.” It is used as a means for introducing chemicals, DNA, or other materials into cells.

Irreversible electroporation (IRE) uses electrical pulses delivered by one or more probes to a targeted area of tissue to produce high amplitude electric fields to serve as the active means for tissue destruction by a specific means, i.e. by fatally disrupting the cell membrane. Davalos, R. V. and Rubinsky, B., (2008) International Journal of Heat and Mass Transfer, 51(23-24), 5617-5622. IRE also encompasses the permeabilization of the cell membrane through the application of electrical pulses across the cell. However, in IRE the permeabilization of the cell membrane does not cease after the application of the pulse and the cell membrane permeability does not revert to normal. The cell does not survive IRE and the cell death is caused by the disruption of the cell membrane and not merely by internal perturbation of cellular components. Openings in the cell membrane are created and/or expanded in size resulting in a fatal disruption in the normal controlled flow of material across the cell membrane. The cell membrane is highly specialized in its ability to regulate what leaves and enters the cell. IRE destroys that ability to regulate in a manner such that the cell can not compensate and as such the cell dies. IRE is a form of nonthermal ablation that does not cause damage to proteins such as, but not limited to, collagen, serum albumin, and elastin.

Cryoablation destroys tissue through one or more cycles of localized freezing using low temperatures (e.g., typically below about −40° C.). The low temperatures used in cryoablation induce local tissue necrosis. In some aspects, the cryoablation involves cycles of freezing and thawing of the tissue to be ablated. Cryoablation of pathological tissues, for example, is typically accomplished by utilizing imaging modalities such as, but not limited to, x-ray, ultrasound, CT, and MRI to identify a locus for ablative treatment, then inserting one or more cryoprobes into that selected treatment locus, and cooling the treatment heads of those cryoprobes sufficiently to cause the tissues surrounding the treatment heads to reach cryoablation temperatures, typically below about −40° C. to about −190° C. The cooled tissues thereby lose their functional and structural integrity. Cancerous cells, for example, that have undergone cryoablation cease growing and multiplying, and the cryoablated tumor tissue material, whether from malignant tumors or from benign growths, is subsequently absorbed by the body. Kunkle, D. A. and Uzzo, R. G., (2008) Cancer 113 (10), 2671-2680.

Sources of energy that can be used with IRE and/or cryoablation include, but are not limited to, radiofrequency, microwave, laser, high-intensity focused ultrasound (HIFU), and ultrasound waves. In one aspect, the energy used in tissue ablation of the methods of the technology can be delivered by one or more probes, needles, and/or electrodes each of varying lengths suitable for the particular type of tissue undergoing ablation. For example, electrodes can be made using a variety of materials, sizes, and shapes known in the art, and may be spaced at an array of distances from one another. Conventionally, electrodes have parallel tines and are square, oval, rectangular, circular, or irregular shaped; having a distance of 0.5 to 10 centimeters (cm) between two electrodes; and a surface area of 0.1 to 5 cm². The specifics of the IRE or cryoablation device used in the methods of the technology, including, for example, the types of probes, needles, or electrodes, can be performed with a wide range of variations that are either well known in the art and can be readily modified by a person of ordinary skill in this field depending upon the circumstances and the particular type of tissue undergoing ablation. In some aspects, the probe used in tissue ablation of the methods of the technology includes more than just the energy emitting system, i.e., it can include a lumen through which a fluid can flow.

Complications Arising from Tissue Ablation

Tissue ablation results in damage of the ablated tissue. In one aspect, the damage is cell death to one or more cells of the ablated tissue. In one aspect, the damage is necrosis. In one aspect, the necrosis is coagulative necrosis. With coagulative necrosis, cellular damage centers on protein coagulation of cystosolic and mitochondrial enzymes and nucleic acid-histone protein complexes. Ahmed, M. and Goldberg, S. N., Tumor ablation: principles and practice, Chapter 3, pg. 27, VanSonnenberg, E., McMullen, W., and Solbiati, L., eds., Springer Science+Business Media, Inc., 2005. In some aspects, the damage is apoptosis of one or more of the cells of the ablated tissue.

During or subsequent to tissue ablation, the damage to the ablated cells can result in the release of the intracellular contents of the ablated cells. The intracellular contents of the ablated cells are released into the surrounding normal tissue and/or the blood. Also, during or subsequent to tissue ablation, the contents of the ablated cells of the ablated tissue are often released into the surrounding normal tissue and/or blood. The contents of the ablated cells include, but are not limited to, active and at least partially intact proteins, including enzymes. The released intracellular content (e.g., active proteins, e.g., enzymes) can be harmful to a mammal that has undergone or is undergoing tissue ablation. Release of the ablated cells' contents can lead to hematologic, metabolic, and renal complications in a mammal that is undergoing or has undergone tissue ablation.

As used herein, a “hematological and metabolic complication” refers to a consequence of tissue ablation. In some aspects, hematological complications include, but are not limited to, bleeding and hemorrhage. Bleeding and hemorrhage can result, for example, from widespread clotting as in disseminated intravascular coagulation (DIC) that is a pathological activation of coagulation mechanisms. In DIC, the processes of coagulation and fibrinolysis are dysregulated.

In other aspects, metabolic complications include, but are not limited to, tumor lysis syndrome, tumor seeding, infection, inflammation, multiple organ dysfunction syndrome (MODS), and systemic inflammatory response syndrome (SIRS). The term “tumor lysis syndrome” refers to a group of metabolic complications that can occur, for example, after tissue ablation as treatment for a cancer such as, but not limited to, a solid tumor. In one aspect, the complications are caused by the break-down products of ablated cells. In some aspects, the complications are caused by the release of the intracellular contents of ablated cells. The group of metabolic complications include, but are not limited to, hyperkalemia, hyperphosphatemia, hyperuricemia, hyperuricosuria, hypocalcemia, acute renal failure, and acute uric acid nephropathy.

In some aspects, the hematological and metabolic complications treated by the methods of the technology are caused by a hematological complication that subsequently gives rise to a metabolic complication. For example, bleeding and/or DIC can result in MODS.

Methods for Reducing Complications with Tissue Ablation

The subject technology provides methods of reducing a risk of hematological and metabolic complications in a mammal undergoing tissue ablation. As discussed in the foregoing comments, ablated cells can release their intracellular contents into the surrounding tissue and/or blood that can lead to hematological and metabolic complications. The methods of the technology disclosed herein can reduce the risk of such complications by denaturing the intracellular contents that have been released from the ablated cells either through energy delivered through a probe, needle, or electrode or through the administration of a composition comprising, for example, hypertonic saline or a denaturing agent.

In one aspect, the technology provides a method of reducing a risk of hematological and metabolic complications in a mammal undergoing tissue ablation. The method can include or comprise the steps of inserting a first probe into a mammal and ablating abnormal tissue in the mammal by emitting a first amount of energy from the first probe.

Further, in accordance with some embodiments, the method can be implemented such that after emitting the first amount of energy, proteins released from cells in the abnormal tissue are denatured by emitting a second amount of energy from at least one of the first probe or a second probe inserted into the mammal.

Furthermore, some embodiments can be implemented such that during or after the emitting the energy from a probe, a composition is administered to the mammal in an effective amount to denature proteins released from cells in the abnormal tissue.

Additionally, some embodiments of methods disclosed herein can denature abnormal tissue by both emitting a second amount of energy and administering a composition to the mammal.

In some aspects, the ablation used in methods of the technology is IRE. In other aspects, the ablation used in methods of the technology is cryoablation. In some aspects, methods of the technology use IRE use probes, needles, and/or electrodes to deliver a desired amount of energy to ablate abnormal and/or undesired tissue. In some aspects, methods of the technology that use cryoablation use probes to cool tissue sufficiently to kill cells. In some aspects, methods of the technology that use cryoablation use probes to freeze tissue sufficiently to kill cells. The probes, needles, and/or electrodes, for example, may be introduced into the mammal endoscopically to the tissue treatment region by passing the electrodes through the working channel of an endoscope or other suitable ablation device that would be readily known a person of ordinary skill in this art.

Ablation devices used in embodiments of the methods disclosed herein can comprise one or more probes that can be positioned in a tissue treatment region of a mammal endoscopically. In some aspects, in methods of the technology that use cryoablation, the probe or needle used to cool abnormal and/or undesired tissue may serve as, or be converted into, a probe, needle, and/or electrode that delivers microwave energy or radiofrequency energy. The resultant dual-purpose or convertible probe may be used to cryoablate tissue and also to denature intracellular material released by cryoablated cells.

In some aspects, methods of the technology can further comprise monitoring the heating of a region of tissue comprising the abnormal tissue that has undergone or is undergoing ablation during and/or after emitting the first amount of energy.

Additionally, the monitoring of heating may be conducted on a region of tissue comprising the abnormal tissue during and/or the emitting of a second amount of energy. In one aspect, the monitoring of the heating is performed with a temperature sensor placed in the mammal. In one aspect, the proteins released from cells in the abnormal tissue are denatured when the second amount of energy reaches about 42° C. to about 60° C. for at least one hour. In some aspects, the proteins released from cells in the abnormal tissue are denatured instantaneously when the second amount of energy reaches about 62° C. or greater. In one aspect, the monitoring is performed by imaging with at least one of CT or MRI.

In some aspects, the steps of ablating abnormal tissue in the mammal by emitting the first amount of energy from the first probe and of denaturing the proteins released from cells in the abnormal tissue by emitting the second amount of energy in the methods of the technology may be repeated one or more times to achieve the desired reduced risk of hematological and metabolic complications in a mammal undergoing tissue ablation. In other aspects, there is a period of time between repeated steps. An appropriate period of time between repeated steps may be, for example, between about one minute and about 35 minutes (e.g., two minutes, three minutes, etc.). An appropriate period of time may also be between about one minute and about three hours. In some embodiments, an appropriate period time may be up to several hours. The period of time may be determined by the physician based on the procedure and the progress of the patient.

In other aspects, the steps of ablating abnormal tissue and of denaturing the proteins released from cells in the abnormal tissue of methods of the technology may be alternated or performed in an alternating manner to achieve the desired reduced risk of hematological and metabolic complications in a mammal undergoing tissue ablation. In other aspects, there is a period of time between performance of the steps of ablating abnormal tissue and of denaturing the proteins released from cells in the abnormal tissue of methods of the technology.

In some aspects, the technology also provides a method of reducing a risk of hematologic and metabolic complications in a mammal undergoing tissue ablation, comprising: inserting a probe into a mammal; ablating abnormal tissue in the mammal by emitting energy from the probe; and during or after the emitting, administering a composition to the mammal that denatures proteins released from cells in the abnormal tissue.

In some aspects, the steps of ablating abnormal tissue in the mammal by emitting the energy from the probe and administering a composition to the mammal that denatures proteins released from cells in the abnormal tissue in the methods of the invention may be repeated one or more times to achieve the desired reduced risk of hematological and metabolic complications in a mammal undergoing tissue ablation. In other aspects, there is a period of time between repeated steps. The period of time may be, for example, between about one minute and about thirty-five minutes (e.g., two minutes, three minutes, etc.). An appropriate period of time may also be between about one minute and about three hours. As similarly noted above, in some embodiments, an appropriate period time may be up to several hours. The period of time may be determined by the physician based on the procedure and the progress of the patient.

In other aspects, the steps of ablating abnormal tissue and of administering a composition to the mammal administering a composition to the mammal that denatures proteins released from cells in the abnormal tissue of methods of the invention may be alternated or performed in an alternating manner to achieve the desired reduced risk of hematological and metabolic complications in a mammal undergoing tissue ablation. In other aspects, there is a period of time between performance of the steps of ablating abnormal tissue and of administering a composition to the mammal of methods. The period of time may be within the ranges noted above.

In one aspect, the composition can include saline that is hypertonic relative to the concentration of saline in a mammal's circulation. In some aspects, the composition includes a denaturing agent. A denaturing agent may comprise, for example, one or more chaotropic agent(s), lyotropic agent(s), organic denaturant(s), and/or detergent(s).

Preferably, in those aspects wherein the denaturing agent includes a detergent, the denaturing agent also includes one or more chaotropic agent(s), lyotropic agent(s), and/or organic denaturant(s), e.g., the denaturing agent further comprises a detergent, in addition to a chaotropic agent, lyotropic agent and/or organic denaturant. Chaotropic agents may include a variety of different compounds, such as, for example, urea, CNS⁻, and CCl₃COO⁻, guanidine HCl (GuHCl), NO₃⁻, and ClO₄⁻. Lyotropic agents may include, for example, SO₄²⁻, HPO₄²⁻, and acetate (CH3COO⁻), e.g., sodium acetate (NaOAc). Organic denaturants may include, for example, acetonitrile (ACN). Detergents may include anionic, cationic, nonionic, or zwitterionic, detergent(s). Examples of denaturing agents include: urea, a zwitterionic detergent (e.g., CHAPS), cetyl trimethyl ammonium bromide (CTAB), GuHCL, acetonitrile, and acetate.

In some aspects wherein at least one denaturing agent is urea, the urea has a concentration of at least about 0.8M, or at least about 1M, when placed in contact with the ablated tissue or tissue that will be ablated, or in proximity to the ablated tissue. For example, the urea may have a concentration in the range of from about 1M to about 9 M, or in the range of from about 1M to about 6M, when placed in contact with the ablated tissue or tissue that will be ablated, or in proximity to the ablated tissue.

In some embodiments wherein at least one denaturing agent is CHAPS, the CHAPS has a concentration of at least about 0.1%, or at least about 0.25%, when placed in contact with the ablated tissue or tissue that will be ablated, or in proximity to the ablated tissue. For example, the CHAPS may have a concentration in the range of from about 0.25% to about 2% when placed in contact with the ablated tissue or tissue that will be ablated, or in proximity to the ablated tissue.

In some embodiments wherein at least one denaturing agent is GuHCl the GuHCl has a concentration of at least about 0.03M, or at least about 0.05M, when placed in contact with the ablated tissue or tissue that will be ablated, or in proximity to the ablated tissue. For example, the GuHCl can have a concentration in the range of from about 0.05M to about 2M when placed in contact with or in proximity to the ablated tissue or tissue that will be ablated. In some embodiments, the GuHCl can have a concentration in the range of from about 0.1M to about 1M when placed in contact with or in proximity to the ablated tissue or tissue that will be ablated.

In some embodiments wherein at least one denaturing agent is acetonitrile, the acetonitrile has a concentration of at least about 8%, or at least about 10%, when placed in contact the ablated tissue or tissue that will be ablated, or in proximity to the ablated tissue. For example, the acetonitrile can have a concentration in the range of from about 10% to about 40% when placed in contact with or in proximity to the ablated tissue or tissue that will be ablated. In some embodiments, the acetonitrile can have a concentration in the range of from about 20% to about 30% when placed in contact with or in proximity to the ablated tissue or tissue that will be ablated.

In some embodiments wherein at least one denaturing agent is acetate, the acetate has a concentration of at least about 30 mM, or at least about 50 mM, when placed in contact with the ablated tissue or tissue that will be ablated, or in proximity to the ablated tissue. For example, the acetate can have a concentration in the range of at least about 50 mM to about 200 mM when placed in contact with or in proximity to the ablated tissue or tissue that will be ablated. In some embodiments, the acetate can have a concentration in the range of at least about 80 mM to about 150 mM when placed in contact with or in proximity to the ablated tissue or tissue that will be ablated. Any appropriate concentration of a denaturing agent known to those of skill in the art may be selected to accomplish sufficient denaturation.

Denaturing agents can be used individually, sequentially, or in combination, e.g., two or more agents sequentially, or in combination, in some embodiments, three or more agents sequentially, or in combination. For example, in one aspect utilizing at least two agents, a chaotropic agent (e.g., urea) is utilized in combination with a detergent (e.g., CHAPS), in a buffer. The concentration of denaturing agent(s) placed in contact with the target molecule optionally may be adjusted to optimize the denaturation of intracellular contents that have been released by ablated tissue or tissue that will be ablated.

In some aspects, the composition, which includes, for example, hypertonic saline or a denaturing agent, may be heated. In some aspects, the composition of the methods is administered in a probe, needle, and/or electrode independently from the probe, needle, and/or electrode used to deliver the energy used to ablate abnormal and/or undesired tissue in a mammal. In some aspects, a probe of an ablation device used in the methods of the technology can include a lumen through which the composition of the method can flow. In other aspects, a the composition administered in the methods of the technology can flow through a lumen in a probe that also emits energy used to ablate abnormal tissue in a mammal.

In one aspect, the method can comprise administering a composition that also includes infusing a contrast agent into the mammal. For example, the contrast agent can be administered with hypertonic saline. Any of a variety of contrast agents can be used which enhance the visibility of cells of tissue during a procedure such as tissue ablation.

There are many means for visualizing tissue undergoing or that has undergone tissue ablation. For example, tissue may be visualized using MRI, ultrasound, nuclear imaging, PET scanning, or photolabels used in optical imaging. Contrast agents may be administered orally or intravenously.

Contrast agents may comprise paramagnetic metal chelates (e.g., Gd, Fe (including iron oxide (e.g., superparamagnetic iron oxide and ultrasmall superparamagnetic iron oxide), Mn (e.g., Mn-DPDP), Cr, Cu, and Eu) for use in MRI scanning. The organic chelator has polar groups that help to act as a ligand, or bridge, between the protein and paramagnetic agent. These chelators, which are well established in the art, include, but are not limited to DTPA, EDTA and TETA. Contrast agents also may comprise materials that are useful for ultrasound that are echogenic, and include, but are not limited to various gases and natural and synthetic materials. Contrast agents may also include radioactive agents. These materials are well described and established in the art. In some aspects, the contrast agent may be heated.

In some aspects, the method of the technology can further comprise monitoring the denaturation of proteins released from a region of tissue comprising the abnormal tissue that has undergone or is undergoing ablation during and/or after emitting an amount of energy.

Additionally, the monitoring of denaturation of proteins released from on a region of tissue comprising the abnormal tissue may be conducted during and/or the administration of the composition that comprises, for example, hypertonic saline or a denaturing agent. In one aspect, the monitoring of denaturation of proteins released from ablated cells is performed by imaging with at least one of CT or MRI.

In some aspects, the composition of the methods of the technology is administered by any means known to those of ordinary skill in this field. In some aspects, the composition is administered by separate needles, introducers used to insert probes, or ports in the IRE or cryoablation probe, needle, and/or electrode.

Although embodiments of these inventions have been disclosed in the context of certain examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combinations or sub-combinations of specific features and aspects may be made and still fall within the scope of the inventions. It should be understood that various features and aspects of the disclosed embodiments can be combined with or substituted for one another in order to form varying modes of the disclosed inventions.

What is claimed is:

1. A method for reducing a risk of hematologic and/or metabolic complications in a mammal undergoing tissue ablation, comprising:
inserting a first probe into a mammal;
ablating abnormal tissue in the mammal by emitting a first amount of energy from the first probe; and
after emitting the first amount of energy, denaturing proteins released from cells in the abnormal tissue by emitting a second amount of energy from at least one of the first probe or a second probe inserted into the mammal.

2. The method of claim 1, further comprising monitoring heating of a region of tissue comprising the abnormal tissue.

3. The method of claim 2, wherein the monitoring heating occurs during emission of the first amount of energy.

4. The method of claim 2, wherein the monitoring is performed with a temperature sensor placed in the mammal.

5. The method of claim 2, wherein the monitoring is performed by imaging with at least one of x-ray, ultrasound, computed topography (CT) or magnetic resonance imaging (MRI).

6. The method of claim 2, wherein the monitoring heating occurs during emission of the second amount of energy.

7. The method of claim 1, wherein the second amount of energy is emitted at least until the temperature of a region of tissue reaches about 42° C. to about 60° C. such that proteins released from cells in the abnormal tissue can be denatured.

8. The method of claim 7, wherein the second amount of energy is emitted until the temperature of the region of tissue reaches about 42° C. to about 60° C. for at least one hour.

9. The method of claim 1, wherein the second amount of energy is emitted such at least until the temperature of a region of tissue reaches about 62° C.

10. A method of reducing a risk of hematologic and metabolic complications in a mammal undergoing tissue ablation, comprising:
inserting a probe into a mammal;
ablating abnormal tissue in the mammal by emitting energy from the probe; and
administering a composition to the mammal in an effective amount to denature proteins released from cells in the abnormal tissue.

11. The method of claim 10, wherein the administering occurs during the emitting.

12. The method of claim 10, wherein the composition is administered through the probe.

13. The method of claim 10, wherein the administering comprises infusing a contrast agent into the mammal.

14. The method of claim 13, wherein the contrast agent is heated before the administering.

15. The method of claim 13, wherein the contrast agent is administered with saline, wherein with saline is hypertonic relative to a concentration of saline in the mammal's circulation.

16. The method of claim 10, wherein the composition comprises a denaturing agent.

17. The method of claim 16, wherein the denaturing agent comprises at least one of a chaotropic agent, a lyotropic agent, an organic denaturant, or a detergent.

18. The method of claim 17, wherein the chaotropic agent is urea.

19. The method of claim 10, further comprising monitoring the denaturation of proteins released from a region of tissue comprising the abnormal tissue.

20. The method of claim 10, wherein the effective amount is effective to reduce a risk in a mammal of at least one of disseminated intravascular coagulation (DIC), tumor lysis syndrome, infection, bleeding, hemorrhage, tumor seeding, or multisystem organ failure.
